(19) United States
(12) Patent Application Publication
(10) Pub. No.: US 2023/0149456 A1
(43) Pub. Date: May 18, 2023

(54) NANOPARTICLE-LOADED SILICIFIED CELLS, METHODS OF MAKING, AND METHODS OF USE

(71) Applicants: Rita E. Serda, Albuquerque, NM (US); Jimin Guo, Albuquerque, NM (US); Achraf Noureddine, Albuquerque, NM (US); Stefan A. Franco, Albuquerque, NM (US); Henning DeMay, Albuquerque, NM (US); UNM RAINFOREST INNOVATIONS, Albuquerque, NM (US)

(72) Inventors: Rita E. Serda, Albuquerque, NM (US); Jimin Guo, Albuquerque, NM (US); Achraf Noureddine, Albuquerque, NM (US); Stefan A. Franco, Albuquerque, NM (US); Henning DeMay, Albuquerque, NM (US)

(21) Appl. No.: 17/916,193

(22) PCT Filed: Mar. 30, 2021
(86) PCT No.: PCT/US2021/024776

§ 371 Date: Sep. 30, 2022

Related U.S. Application Data
(60) Provisional application No. 63/001,737, filed on Mar. 30, 2020.

(51) Int. Cl. A61K 35/13; B82Y 5/00; A61K 35/74; A61K 35/76

(52) U.S. Cl. CPC A61K 35/13; B82Y 5/00; A61K 35/74; A61K 35/76

(57) ABSTRACT

A silicified cell includes a nanoparticle that carries a bioactive agent. The silicified call can be a tumor cell, a bacterial cell, a virus, or a silicifiable compartment or fragment thereof. The silicified cell can optionally include an immunomodulatory moiety that may be carried within pores of the nanoparticle and/or bound to the surface of the nanoparticle. The silicified cell can be used as a prophylactic or therapeutic treatment for treating tumors or bacterial infections.

### CROSS-REFERENCE TO RELATED APPLICATION

This application claims the benefit of U.S. Provisional Patent Application No. 63/001,737, filed Mar. 30, 2020, which is incorporated herein by reference in its entirety.

### SUMMARY

This disclosure describes, in one aspect, a silicified cell that includes a nanoparticle that includes a bioactive agent. The silicified cell can be a tumor cell, a bacterial cell, a virus, an embryonic cell, a fetal cell, a pluripotent stem cell (e.g., an induced pluripotent stem cell), or a silicifiable compartment or fragment thereof. The silicified cell can optionally include an immunomodulatory moiety that may be carried within pores of the nanoparticle and/or bound to the surface of the nanoparticle.

In some embodiments, the bioactive agent can include a chemokine, a cytokine, a growth factor, a chemotherapeutic, an anti-angiogenic factor, an antibody, a DAMP, a PAMP, a DNA plasmid, an siRNA, an mRNA, or a combination thereof.

In some embodiments, the immunomodulatory moiety can include a pathogen-associated molecular pattern (PAMP), a danger-associated molecular molecule (DAMP), a cytokine, an antibody, or a combination thereof. In some of these embodiments, the PAMP can include lipopolysaccharide (LPS), monophosphoryl lipid A (MPL), CpG, R-848, PolyIC, or any combination thereof.

In another aspect, this disclosure describes a method of preparing a silicified cell. Generally, the method includes obtaining a cell, loading the cell with a nanoparticle comprising a bioactive agent, and silicifying the cell.

In another aspect, this disclosure describes a method of inducing an immune response against a cell. Generally, the method includes obtaining a cell, loading the cell with a nanoparticle that carries a bioactive agent, silicifying the cell thereby producing an immunogenic silicified cell, and administering the immunogenic silicified cell to a subject in an amount effective to induce the subject to produce an immune response directed against the cell.

In some embodiments, the cell can be a tumor cell, a bacterial cell, a virus, a fetal cell, an embryonic stem cell, or an induced pluripotent stem (IPS) cell.

In some embodiments, the nanoparticle includes an agent that blocks immune suppression.

In another aspect, this disclosure describes a method for treating a subject having, or at risk of having, a tumor. Generally, the method includes obtaining a tumor cell that the subject has or is at risk of having, loading the tumor cell with a nanoparticle carrying a bioactive agent, silicifying the tumor cell, and administering the silicified tumor cell to the subject in an amount effective to ameliorate at least one symptom or clinical sign of having the tumor.

In some embodiments, the tumor call may be autologous. In other embodiments, the tumor call may be allogenic.

In another aspect, this disclosure describes a method for treating a subject having, or at risk of having, a bacterial infection. Generally, the method includes obtaining a bacterial cell that the subject is, or is at risk, of being infected by, loading the cell with a nanoparticle carrying a bioactive agent, silicifying the bacterial cell, and administering the silicified bacterial cell to the subject in an amount effective to ameliorate at least one symptom or clinical sign of infection by the bacterial cell.

In some embodiments, the bacterial cell is obtained from the subject.

In another aspect, this disclosure describes a method for treating a subject having, or at risk of having, a viral infection. Generally, the method includes obtaining a virus that the subject is, or is at risk, of being infected by, loading the virus with a nanoparticle carrying a bioactive agent, silicifying the virus, and administering the silicified virus to the subject in an amount effective to ameliorate at least one symptom or clinical sign of infection by the virus.

In some embodiments, the virus is obtained from the subject.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1. Modular personalized cancer vaccines. Schematic showing nanoparticle (NP) uptake by cancer cells, followed by cell biomineralization (silicification), and then uptake by dendritic cells.

FIG. 2. 3D confocal micrographs of dendritic cells (red; actin) following uptake of nanoparticles (green) loaded silicified cancer cells. Nuclei are shown in blue (DAPI).

FIG. 3. Scanning electron micrographs (grayscale or false-colored) capture direct cell-to-cell transfer of a cluster of silica nanoparticles.

FIG. 4. Direct cell-to-cell connections and nanoparticle exchange. Scanning electron microscopy (SEM) images show mixed cultures of RAW macrophages and HeLa cells.

FIG. 5. RAW and HeLa cells were preloaded with fluorescent nanoparticles (distinct fluorophores) and then mixed to evaluate the amount of nanoparticle exchange between cells using flow cytometry.

FIG. 6. Cy3-siRNA loading efficiency in lipid coated MSN (LC-MSN). 50 mg LC-MSN were loaded with 5 μg/mL, 20 μg/mL, or 50 μg/mL Cy3-siRNA and encapsulation efficiency was calculated by measuring siRNA remaining in the loading supernatant following removal of siRNA-loaded LC-MSN.

FIG. 7. Assembly of a modular personalized cancer vaccine. Top: Schemtic illustration showing assembly of silificied cancer cells loaded with siRNA-nanoparticles and coated with TLR ligands. Bottom left: Aminis Imagestream dotplot shows the proportion of silicified floureoscent (GFP) 4T1 breast cancer cells that have internalized Cy3-siRNA/DyLight 633 LC-MSN (upper right region). Bottom middle: Images of independent and merged flourescent channels support the assembly of single entities containing drug (siRNA), nanoparticles, and silicified cancer cells. Bottom right: Pie chart shows the proportion of silicified GFP-4T1 cells that contain Cy3-siRNA loaded DyLight 633 LC-MSN; flow cytometry data showing untreated 4T1 GFP cells, 4T1 cells following incubation with Cy3-siRNA loaded DyLight 633 LC-MSN, and silicified 4T1 cells following incubation with Cy3-siRNA loaded DyLight 633 LC-MSN.

FIG. 8. BMDC internalization of a modular personalized cancer vaccine. Flow cytometry dotplots showing gating used to select CD11c+ BMDC, which were subsequently examined for dual expression of Cy3-siRNA and DyLight 633-LC-MSN indicative of internalization of a fully loaded modular vaccine.

FIG. 9. BMDC internalization of a modular personalized cancer vaccine. Bar graph showing percent positive BMDC.

### DETAILED DESCRIPTION OF ILLUSTRATIVE EMBODIMENTS

This disclosure describes a personalized vaccine platform that involves nanoparticle-loaded silicified cells. The silicified cells are biomineralized cells—e.g., tumor cells—against which an immune response is desired. The immunogenicity of the silicified cells is enhanced by loading the cells with nanoparticles that contain a bioactive agent prior to the cell being silicified.

Dendritic cells (DC) are potent antigen presenting cells. Under homeostatic conditions, a mixed population of immature DCs resides in tissues and cavities throughout the body, including the peritoneal cavity. Their precise location is regulated by a variety of chemotactic and other signals, including bacterial products (i.e., pathogen-associated molecular patterns, PAMPs), danger-associated molecular patterns (DAMPs), complement factors, and lipids. Chemokines attracting dendritic cells to lymphoid organs include CCL19, CCL21, CXCL12, MIP-la, and MIP-5. Once activated, the dendritic cells secrete cytokines that attract T cells and activate innate and adaptive effector cells. In patients with cancer, immune suppression overwhelms immune-mediated elimination of cancer cells and enables cancer progression.

Biomineralization can transform cells into stable, immunogenic entities. Biomineralized cells can be functionalized to display microbial molecules on the surface, making the biomineralized cells microbe mimetics, able to stimulate potent immune responses.

Following intraperitoneal injection of biomineralized ovarian cancer cells into mice with ovarian cancer, dendritic cells and other phagocytic immune cells internalize the biomineralized cancer cells, leading to DC activation and antigen presentation to T cells. Cytolytic T cells then kill cancer cells. Biomineralized cells and vaccines prepared from biomineralized cells are described in, for example, International Publication No. WO 2019/055620, U.S. Patent Publication No. US 2020/0276286 A1, and International Patent Publication No. WO 2020/020776 A1.

This disclosure describes a modular immunotherapy platform based on immunogenic silicified cells that further enhance an immune response against a biomineralized cellular target (e.g., a cancer cell or an infectious microbe). The modular immunotherapy platform described herein supports one or more of the following: immune cell attraction via release of chemokines; immune cell activation via cytokine releases (e.g., IL-12); alleviation of immune suppression (e.g., anti_PD-1, anti-PD-L1, anti-CTLA-4, anti-TIM3, anti-LAG3, anti-CD47, and/or an IDO inhibitor); gene silencing (e.g., TGF-beta, IL-10, or PDL1 siRNA); gene expression (e.g., IL-12 mRNA); delivery of chemotherapeutics, small molecules, or other targeted drugs; delivery of anti-angiogenic molecules to normalize tumor vasculature; delivery of biomimics to alter the phenotype and/or function of targeted cells; or delivery of catabolites and metabolites to alter energy balance and oxygen consumption of target cells.

The modular immunotherapy platform described herein involves preloading cells with cargo-carrying nanoparticles prior to cell silicification, creating immunogenic silicified cells that work at multiple levels. While the biomineralized cell vaccines stimulate immune responses against the biomineralized cellular target, the additional agents provided in the preloaded nanoparticles can support sustained immune responses by, for example, helping to recondition the tumor microenvironment.

While described below in the context of exemplary embodiments in which the cells being loaded with nanoparticles and then silicified are cells of a cancer cell line, the compositions and methods described herein can involve immunogenic silicified cells prepared from any cell type. Alternative suitable cell types include, for example, any cell type that poses a danger to the host and where an immune response against antigens associated with the cell would benefit the host. This includes all types of cancer cells (autologous or allogeneic), pathogenic cells (e.g., microbes), or cells that express antigens associated with cancer. The latter includes embryonic cells and cells genetically modified to cause expression of tumor antigens or tumor-associated antigens. Thus, exemplary alternative cell types include, but are not limited to, a bacterial cell, an embryonic cell, a fetal cell, or a pluripotent stem cell (e.g., and induced pluripotent stem cell). As noted in more detail below, the vaccine platform need not necessarily involve using an entire cell. In some embodiments, the vaccine platform may involve the use of a silicifiable compartment of a cell such as, for example, an exosome, a vesicle, a spheroid, an organoid, or an organelle. In still other embodiments, as used herein, the term “cell” can include a virus or a virus-like particle (VLP). Even though viruses are not cells and lack many of the structures of cells, viruses (and/or VLPs) can be loaded with nanoparticles (Jeevanandam et al., 2019, Biochemie 157:38-47; Sainsbury, F., 2017, Ther Deliv 8(12):1019-1021). Once loaded with nanoparticles, viruses and/or VLPs can be silicified in the same way that a cell or cellular compartment may be silicified. Thus, in the context of the compositions and methods described herein, viruses and VLPs can act as cells or silicifiable cellular compartments.

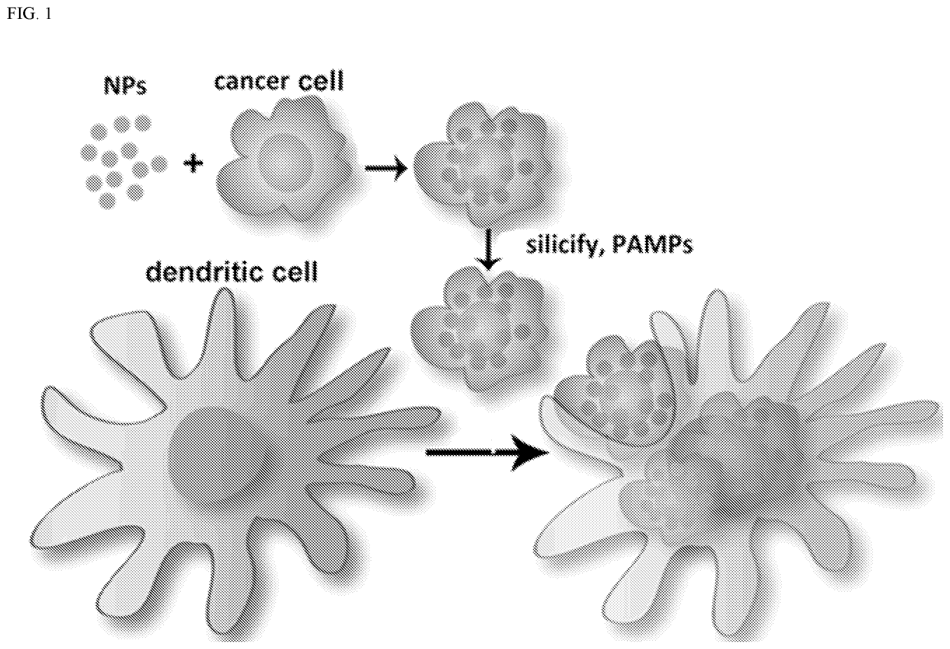

Cancer cell internalization of nanoparticles occurs rapidly, especially when nanoparticles are cationic or have surface moieties that interact with receptors on the plasma membrane of cancer cells. FIG. 1 is a schematic showing cancer cell uptake of nanoparticles (green), followed by cell silicification and coating with PAMPs. Injection of the transformed cancer cells into patients leads to uptake by dendritic cells and presentation of cancer antigens leading to an anti-cancer immune response.

The nanoparticles may be prepared as previously described. (e.g., US Patent Application Publication No. US 2018/0344641; International Publication No. WO 2019/028387; US Patent Application Publication No. US 2020/0009264; and International Publication No. WO 2019/169152).

While sometimes described below in the context of an exemplary embodiment in which the nanoparticle is a mesoporous silica nanoparticle (MSN), the compositions, platform, and methods described herein can involve any suitable form of nanoparticle. Exemplary suitable nanoparticles include, but are not limited to, nanoparticles prepared from liposomes, MSNs, silicon, poly lactic-co-glycolic acid) (PLGA), iron oxide (theranostic), gold, gold nanoshells, dendrimers, micelles, biocompatible polymers, etc.

The nanoparticle cargo can include any desired bioactive agent such as, for example, a chemokine, a cytokine, a growth factor, a small molecule, a chemotherapeutic, an anti-angiogenic factor, an antibody, a DAMP, a PAMP, an siRNA, an mRNA, a DNA plasmid, or other proteins or lipids. By loading a single agent per batch of nanoparticles, one can optimize the nanoparticles for each cargo, which can increase loading. A single nanoparticle can, however, include any combination of two or more bioactive agents, which can load the target cell with a combination of bioactive agents. Alternatively, a biomineralized cell can deliver a combination of two or more bioactive agents by being preloaded with two or more population of nanoparticles loaded with different bioactive agents—i.e., a first population of nanoparticles containing a first bioactive agent or combination of bioactive agents, and a second population of nanoparticles containing a second a different bioactive agent or combination of bioactive agents, which differs from the bioactive agent or agents loaded into the first population of nanoparticles.

The modular immunotherapy platform described herein exploits the uptake of nanoparticles by target cells. A target cell may be loaded with one or more nanoparticles. When the target cell is loaded with a plurality of nanoparticles, the population of nanoparticles loaded into the target cell may be homogeneous or heterogeneous. In a homogeneous population of nanoparticles, the composition and cargo of all of the nanoparticles loaded into the cell are identical. In a heterogeneous population of nanoparticles, the composition of the nanoparticles and/or cargo loaded in nanoparticles may differ. Moreover, a single nanoparticle may be loaded with one or more cargo molecules. Mixed cargo, whether provided within a single nanoparticle or provided in a heterogeneous mixture of nanoparticle, can include, for example, an inhibitor of immune suppression and/or an immune stimulant (e.g., IL-12 mRNA). Exemplary inhibitors of immune suppression can include, but are not limited to, immune checkpoint inhibitors. Thus, an inhibitor of immune suppression can include, but is not limited to, an anti-PD-1 antibody or siRNA, an anti-PD-L1 antibody or siRNA, an anti-PD-L2 antibody or siRNA, an anti-TIM3 antibody or siRNA, an anti-CTLA-4 antibody or siRNA, an anti-TGF-β antibody or siRNA, an anti-IL-10 antibody or siRNA, etc.

The immune response generated by dendritic cells can be enhanced by silicifying the nanoparticle-loaded target cell and, optionally, modifying the surface of the silicified cell to display one or more immunomodulatory moieties. Suitable immunomodulatory moieties include, but are not limited to, one or more PAMPs, one or more DAMPs, or one or more alternative immunomodulatory moieties. Exemplary PAMPs include, but are not limited to, lipopolysaccharide (LPS), monophosphoryl lipid (MPL), PolyIC, a TLR agonist (e.g., an imidazoquinoline amine such as, for example, R-848), double-stranded RNA, lipoteichoic acid, peptidoglycan, viruses, and unmethylated CpG. DAMPS are endogenous molecules created upon tissue injury. Exemplary DAMPs include, but are not limited to, heat shock proteins, high mobility group box 1, proteins such as hyaluronan fragments, and non-protein targets such as ATP, uric acid, DNA and heparin sulfate.

The surface of a silicified cell may be modified to facilitate binding of the immunomodulatory moiety by, for example, providing a siloxane functional group, a cationic layer disposed on at least a part of the surface, an anionic layer disposed on at least a part of the surface, or a mixture or combination thereof. Methods for providing surface modifications of silicified cells are described in, for example, International Publication No. WO 2019/055620, U.S. Patent Publication No. US 2020/0276286 A1, and International Patent Publication No. WO 2020/020776.

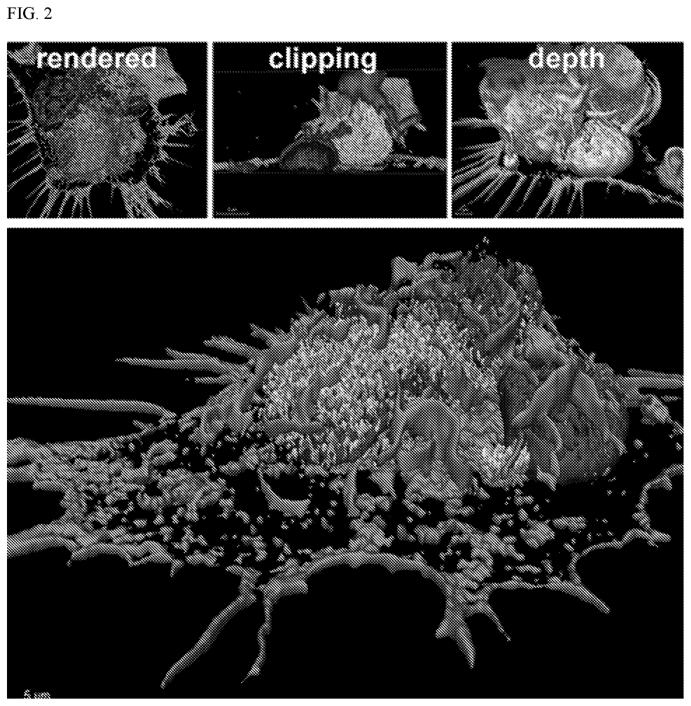

The 3D confocal micrographs in FIG. 2 show a dendritic cell (with the actin cytoskeleton shown in red) with internalized cancer cells (seen as green due to the internalized nanoparticles). In this example, the nanoparticle cargo increased recruitment and activation of immune cells.

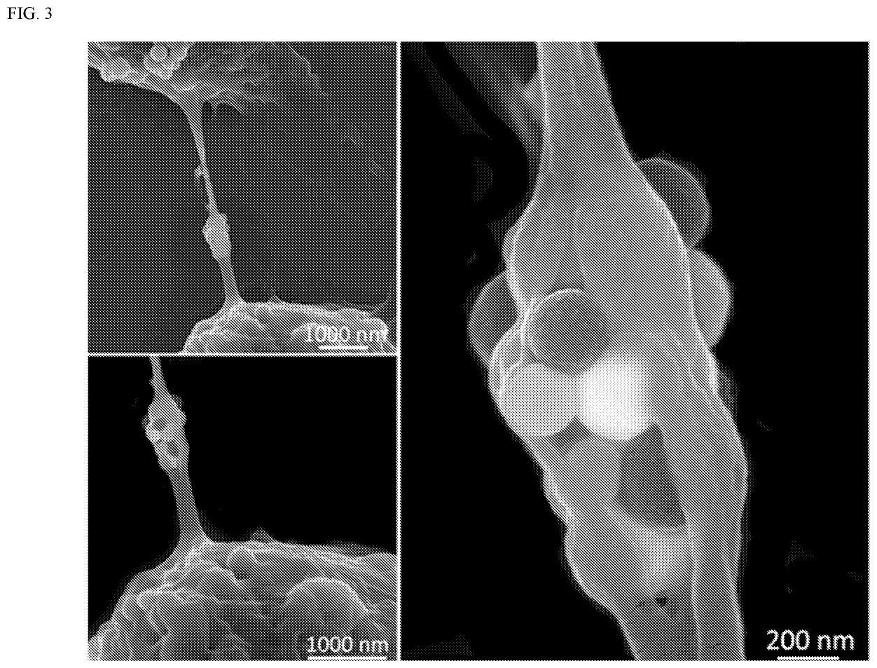

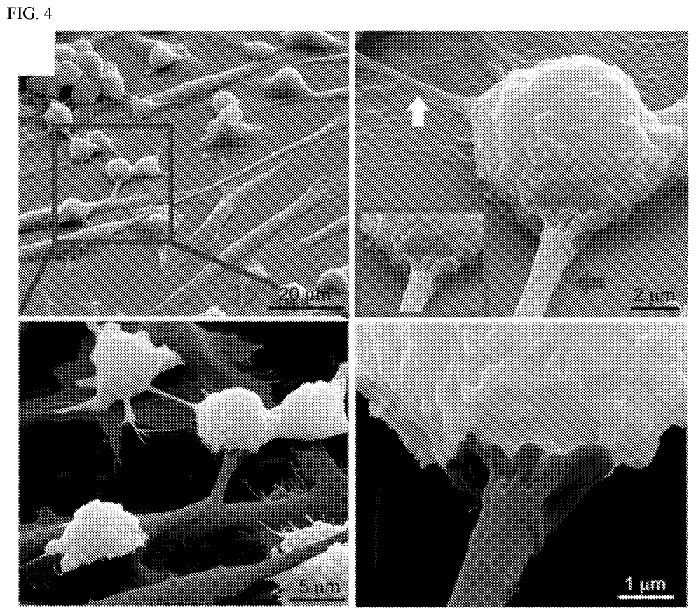

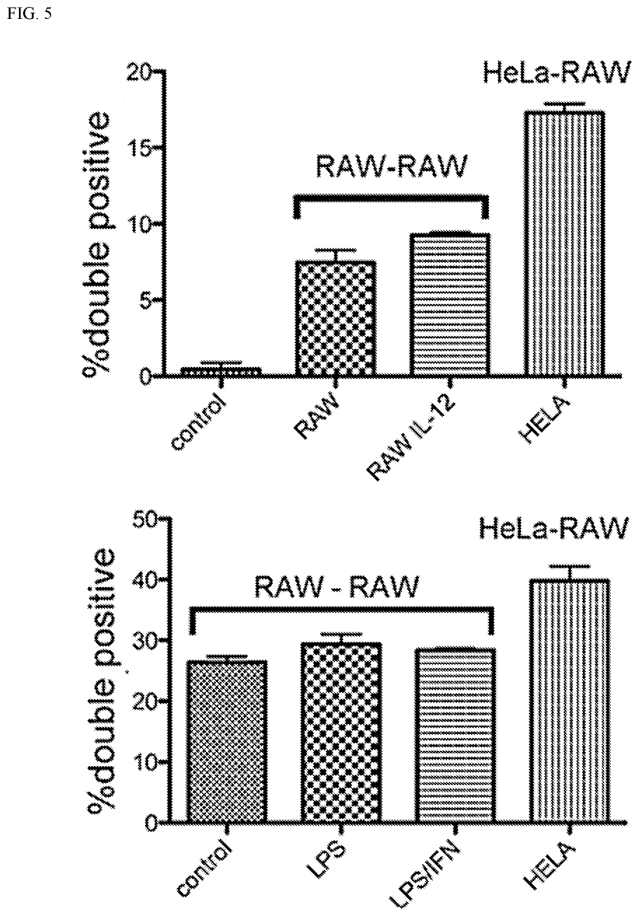

Bioactive agents provided as nanoparticle cargo can work directly on dendritic cells, but can also affect surrounding cells through direct or indirect cell-to-cell transfer. FIG. 3 shows direct cell-to-cell transfer of silica nanoparticles though cellular connections coined tunneling nanotubes. Connections between immune cells and cancer cells is shown in electron micrographs in FIG. 4. In FIG. 5, flow cytometry and fluorescent nanoparticles were used to measure cell-to-cell exchange of nanoparticles. The rate of heterotypic transfer from macrophages (RAW cells) to cancer cells (HeLa) was greater that homotypic transfer between macrophages. Inflammatory factors (e.g., IL-12, LPS, IFN) did not increase the rate of transfer. Alternatively, secretion of nanoparticles or their cargo in exosomes or biovesicles, or in free-form, can lead to uptake by surrounding cells (indirect transfer). Acceptor cells include other immune cells, fibroblasts, endothelia or cancer cells, facilitating activation or reversal of immune suppression, cancer cell death, suppression of angiogenesis, or blockade of checkpoint inhibition, depending on the cargo.

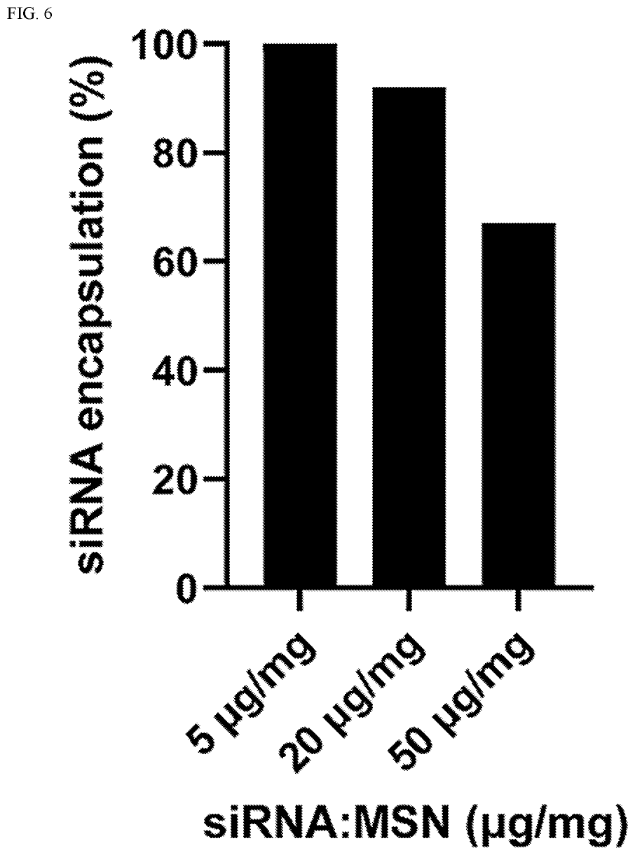

FIG. 6 shows data indicating the efficiency achieved loading an exemplary bioactive agent into an exemplary nanoparticle. Lipid-coated (LC) mesoporous silica nanoparticles were loaded with Cy3-siRNA achieved 100% loading efficiency. When the siRNA was provided at a concentration of 5 μg/mL, 100% loading efficiency was achieved.

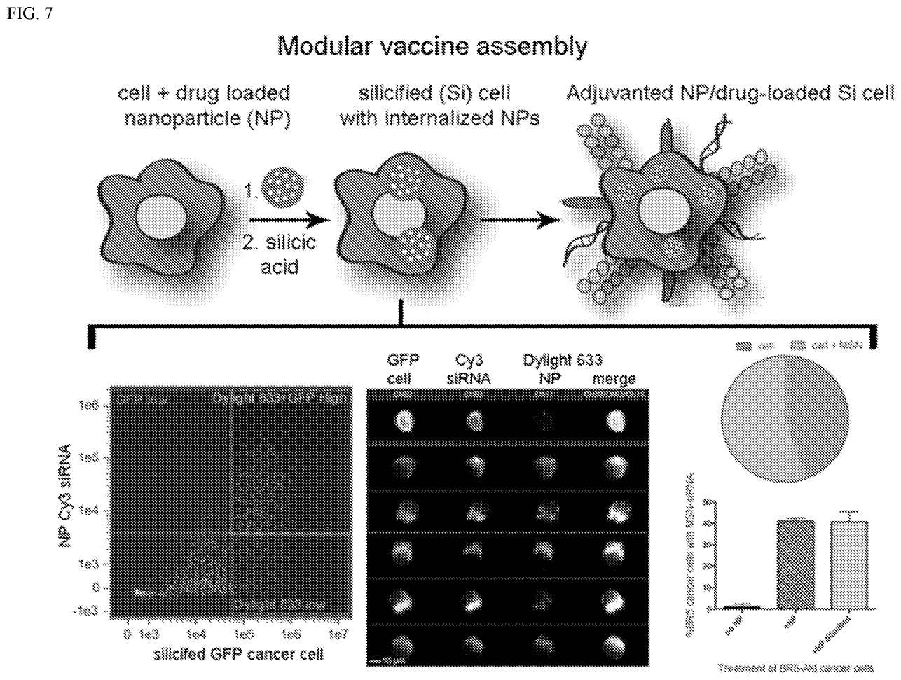

FIG. 7 presents data showing the assembly of an exemplary modular vaccine that includes silicified cancer cells that house drug (siRNA)-loaded nanoparticles (LC-MSN) within the cell and TLR ligands on the silicified cell surface (FIG. 7, top). Briefly, 50 mg of fluorescent nanoparticles (liposome coated DyLight 633 mesoporous silica nanoparticles) were loaded with 50 μg/mL fluorescent nuclei acid (Cy3 siRNA), and then incubated with 20,000 live breast cancer cells [4T1-GFP (green fluorescent protein)] for six hours at 37° C. to facilitate internalization. Following nanoparticle internalization by the tumor cells, the cells were cryo-silicified and then surface coated with TLR ligands (PEI, CpG, and MPL). The composition of the fully assembled vaccine was confirmed using both an imaging cytometer (AMNIS IMAGESTREAM, Luminex Corp., Austin, Tex.; FIG. 7, bottom left and bottom center) and a benchtop flow cytometer (ATTUNE NxT, Thermo Fisher Scientific, Inc., Waltham, Mass.; FIG. 7, bottom, right).

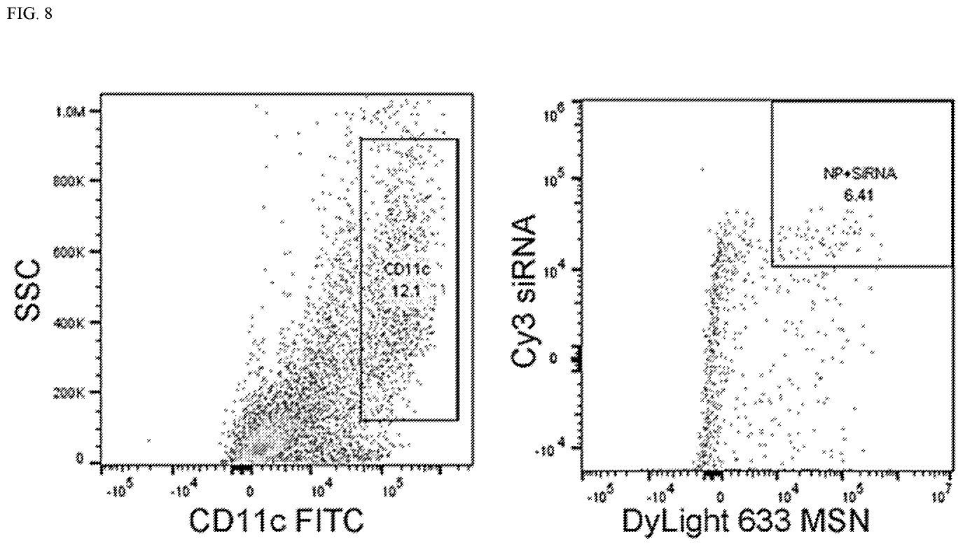

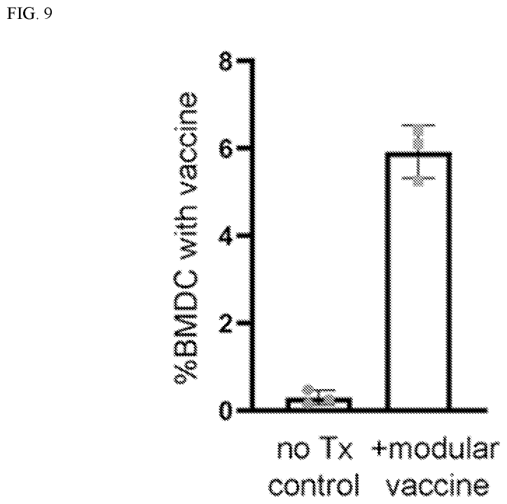

FIG. 8 and FIG. 9 provide flow cytometry dotplot data (FIG. 8) and the same data represented in bar graph form (FIG. 9) showing that CD11c⁺ bone marrow-derived dendritic cells (BMDCs) internalized a modular vaccine of cryo-silicified 4T1-GFP cells containing liposome-coated mesoporous silica nanoparticles loaded with Cy3 siRNA.

An immunogenic silicified cell may therefore be formulated into a pharmaceutical composition. As used herein, “immunogenic silicified cell” refers collectively to a silicified cell or a silicified cell fragment or silicified cell-derived body, such as, for example, a silicified exosome, a silicified microvesicle, or a silicified apoptotic body. Exemplary cells include, but are not limited to, a cell derived from a patient tumor (either autologous or allogenic), blood, ascites, or established tumor cell lines. Additional exemplary cells include, but are not limited to, a bacterial cell, an embryonic cell, a fetal cell, or a pluripotent stem cell (e.g., an induced pluripotent stem cell). The use of embryonic cells, fetal cells, or induced pluripotent stem cells is based on the expression antigens by these cell types that are not normally expressed by normal differentiated cells but may be expressed by cancer cells. Thus, the use of these cells to generate a silicified vaccine can induce an immune response against antigens that are expressed by tumor cells, thereby generating an anti-tumor response. As discussed above, the immunogenic silicified cell can be a silicified virus or virus-like particle (VLP).

The composition may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the pharmaceutical compositions is contemplated. Supplementary active ingredients also can be incorporated into the compositions. As used herein, “pharmaceutically acceptable” refers to a material that is not biologically or otherwise undesirable, i.e., the material may be administered to an individual along with an immunogenic silicified cell without causing any undesirable biological effects or interacting in a deleterious manner with any of the other components of the pharmaceutical composition in which it is contained.

The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical can be administered via a sustained or delayed release.

Thus, an immunogenic silicified cell may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, solution and the like. The formulation may further include one or more additives including such as, for example, an adjuvant. Exemplary adjuvants include, for example, pathogen-associated molecular patterns (PAMPs), such as Toll-like receptor (TLR) ligands, damage-associated molecular patterns (DAMPs), cytokines, proteins, carbohydrates, lectins, Freund's adjuvant, aluminum hydroxide, or aluminum phosphate.

A formulation may be conveniently presented in unit dosage form and may be prepared by methods well known in the art of pharmacy. Methods of preparing a composition with a pharmaceutically acceptable carrier include the step of bringing the immunogenic silicified cell into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of immunogenic silicified cell administered can vary depending on various factors including, but not limited to, the specific silicified cell being administered, the weight, physical condition, and/or age of the subject, and/or the route of administration. Thus, the absolute amount of immunogenic silicified cell included in a given unit dosage form can vary widely, and depends upon factors such as the species, age, weight and physical condition of the subject, and/or the method of administration. Accordingly, it is not practical to set forth generally the amount that constitutes an amount of immunogenic silicified cell effective for all possible applications. Those of ordinary skill in the art, however, can readily determine the appropriate amount with due consideration of such factors.

In some embodiments, the method can include administering sufficient immunogenic silicified cells to provide a dose of, for example, from about 50 silicified cells/kg to about 1×10¹⁰ silicified cells/kg to the subject, although in some embodiments the methods may be performed by administering the immunogenic silicified cells in a dose outside this range. In some of these embodiments, the method includes administering sufficient immunogenic silicified cells to provide a dose of from about 100 silicified cells/kg to 1×10⁹ silicified cells/kg to the subject, for example, a dose of from about 1000 silicified cells/kg to about 10,000 silicified cells/kg.

In some embodiments, immunogenic silicified cells may be administered, for example, from a single dose to multiple doses per month, although in some embodiments the method can be performed by administering immunogenic silicified cells at a frequency outside this range. In certain embodiments, immunogenic silicified cells may be administered from about once every six months to about three times per week.

The silicified cells described herein can be used to treat a subject having, or at risk of having, a condition for which treatment is intended. That is, the treatment may be therapeutic or prophylactic. Treatment that is prophylactic—e.g., initiated before a subject manifests a symptom or clinical sign of the condition for which treatment is intended such as, for example, while an infection remains subclinical—is referred to herein as treatment of a subject that is “at risk” of having the condition. As used herein, the term “at risk” refers to a subject that may or may not actually possess the described risk. Thus, for example, a subject “at risk” of developing a tumor is a subject possessing one or more risk factors associated with developing the tumor such as, for example, genetic predisposition, ancestry, age, sex, geographical location, lifestyle, or medical history. As another example, a subject “at risk” of an infectious condition is a subject present in an area where individuals have been identified as infected by the microbe that causes the condition and/or is likely to be exposed to the microbe that causes the condition even if the subject has not yet manifested any detectable indication of infection by the microbe that causes the condition and regardless of whether the subject may harbor a subclinical amount of the microbe that causes the condition.

Accordingly, a composition can be administered before, during, or after the subject first exhibits a symptom or clinical sign of the condition for which treatment is intended. Treatment initiated before the subject first exhibits a symptom or clinical sign of the condition may result in decreasing the likelihood that the subject experiences clinical evidence of the condition compared to a similarly situated subject to whom the composition is not administered, decreasing the severity of symptoms and/or clinical signs of the condition, and/or completely resolving the condition. Treatment initiated after the subject first exhibits a symptom or clinical sign of the condition for which treatment is intended may result in decreasing the severity of symptoms and/or clinical signs of the condition compared to a similarly situated subject to whom the composition is not administered, and/or completely resolving the condition.

Thus, the method includes administering an effective amount of the composition to a subject having, or at risk of having, a condition for which treatment is intended. In this aspect, an “effective amount” is an amount effective to reduce, limit progression, ameliorate, or resolve, to any extent, a symptom or clinical sign related to the condition.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Synthesis of ˜7 nm Dendritic Pores Monodisperse Mesoporous Silica Nanoparticles

The synthesis of the mesoporous silica nanoparticles was modified from previously reported methods (Noureddine et al., 2019, Journal of Sol-Gel Science and Technology 89(1):78-90).

### Synthesis of Carboxylic Acid-Terminated Mesoporous Silica Nanoparticles (MSN-COOH)

In a 100 mL round bottom flask, triethanolamine (0.18 g) was added along with cetyltrimethylammonium chloride (CTAC, 24 mL, pH=6) and water (36 mL). The pH of the solution was adjusted to 8.5 with sodium hydroxide and the mixture was heated to 50° C. and stirred (600 rpm) for one hour. The stirring rate was adjusted to 350 rpm and a 20 mL solution of TEOS in cyclohexane (10% v/v) was slowly added to form a biphase system. After 16 hours, a solution of triethoxypropylsuccinic anhydride in ethanol (200 μL) was added in the bottom aqueous phase (containing silica nanoparticles) and kept reacting for four hours. The upper organic phase was then removed and the nanoparticles suspension was centrifuged. The isolated pellet is suspended in ethanol and centrifuged twice. The surfactant removal was achieved by successive washing steps by NH₄NO₃ (6 g/L ethanol) and HCl (1% ethanol, twice); each step included 15 minutes sonication and centrifugation. All centrifugation cycles were done at 50,000 relative centrifugal force (rcf) for 20 minutes at 18° C. Lastly, the template-free mesoporous silica nanoparticles were washed twice in ethanol and stored as a suspension in ethanol. The suspension is stable for at least one year.

### Synthesis of Primary Amine-Terminated Mesoporous Silica Nanoparticles (MSN-NH₂)

The procedure was the same as above but 3-aminopropyltriethoxysilane (APTES, 110 μL in 200 μL ethanol) was used as the organosilane.

### Conjugation of Fluorescent Labels on MSN-NH₂

DYLIGHT 800-NHS, or DYLIGHT 488-NHS (Thermo Fisher Scientific, Waltham, Mass.) were used to fluorescently label mesoporous silica nanoparticles. A solution of dye in DMF (1 mg/mL, 250 μL) stored at −20° C. was added to a suspension of MSN-NH₂ (10 mg, 2.5 mg/mL) and reacted for 18-24 h at room temperature in the dark. The mixture was centrifuged and resuspended in succinic anhydride solution in dimethylformamide (DMF, 100 mg, 25 mg/mL) and reacted for 24 hours at room temperature in the dark (in order to turn the charge of the nanoparticles into negative). Next, the mixture was centrifuged and the isolated dyed pellet was washed in DMF (once) then in pure ethanol (thrice). An aliquot was washed in water twice to confirm the final negative charge of the dye-MSN.

### Preparation of Immunogenic Liposomes

Lipids in chloroform (10 to 25 mg/mL) are stored under argon atmosphere at −25° C. A mixture of different lipids formulations was prepared by mixing the corresponding lipids in a glass vial (in a glovebox) with total amounts ranging from 5 mg to 15 mg. The chloroform was removed from the lipid mixture under reduced pressure (rotator evaporator, 10 minutes) then kept under reduced pressure overnight in a vacuum pump in order to remove all chloroform residues. The lipid mixtures were then hydrated in PBS to 5 mg/mL and sonicated for at least 30 minutes at 45° C. The liposomal suspensions were used directly after preparation to form immunogenic lipid-coated MSNs (ILMs).

Ovalbumin Loading Procedure and ILM assembly

A fresh solution of ovalbumin in distilled water (1 or 5 mg/mL) was prepared before the loading procedure. Then, mesoporous silica nanoparticles (dye-labeled or not) in water (1 mg) were incubated (gentle shaking) in the OVA (or other relevant protein) solution (with 1/1 or ⅕/OVA wt ratio) for 15 minutes at room temperature (22° C.) in the dark. Afterwards, on the OVA-MSN mixture, immunogenic liposomes (5 mg) were added under sonication (20 seconds). The obtained mixture was then centrifuged (21,000 rcf, 10 minutes, 4° C.) and the isolated pellet was suspended in PBS (10 mM) and centrifuged. The pellet was resuspended in PBS at 1 mg/mL before in vitro and/or in vivo experiments. All supernatants were saved for protein loading quantitation.

### In Vitro BMDC Internalization of Fluorescent Nanoparticle Loaded Silicified Cells

To image BMDC association with silicified cells, BR5-Akt cancer cells were first incubated with immunogenic (liposome-coated mesoporous silica nanoparticles loaded with MPL) DyLight 488-labeled nanoparticles, respectively, for 1-3 hours prior to cell silicification (using optimized conditions) and surface masking with TLR ligands (as indicated). BMDC were seeded onto glass cover slips in 6-well plates at a density of 5×10⁵ cells per well and the next day, fluorescent silicified vaccine cells were added and BMDC were incubated for an additional 24 hours. BMDC were then washed with PBS and fixed with 4% paraformaldehyde for 15 minutes at room temperature followed by overnight incubation at 4° C. The following day, cells were washed with PBS, permeabilized with 0.1% Triton-X in PBS for 15 minutes, blocked with 1% BSA for 20 minutes, and then labeled with fluorescent phalloidin (Thermo Fisher Scientific, Inc., Waltham, Mass.) in 1% BSA for one hour. After a final wash in PBS, coverslips were mounted on slides using Prolong Gold with DAPI. Images were acquired using a 63×/1.4 NA oil objective in sequential scanning mode using a Leica TCS SP8 confocal microscope.

BMDC uptake of silicified cells was quantified using flow cytometry (ATTUNE NxT flow cytometer; Thermo Fisher Scientific, Inc., Waltham, Mass.) to measure association of fluorescently labeled BMDC and silicified cells.

### Scanning Electron Microscopy (SEM) Imaging of Nanoparticle Transfer

RAW macrophages or HeLa cells were seeded in 24-well plates containing 5×7 mm silicon chip specimen supports (Ted Pella, Inc., Redding, Calif.) at 1×10⁵ cells per well. Cells were then incubated with 10 μg/ml 200 nm silica nanoparticles for 1 hour, 3 hours, or 24 hours, and then processed for SEM imaging as previously described (Serda et al., 2009, Biomaterials 30:2440-2448). Alternatively, HeLa cancer cells were seeded onto silicon chips and the next day RAW cells, preloaded with NPs, were added and cell were incubated for an additional 24 hours.

SEM images were acquired under high vacuum, at 1-30 kV, using a Hitachi SU8230 Scanning Electron Microscope (Hitachi High Technologies, Clarksburg, Md.) or an FEI Quanta 3D FEG, (FEI, Hillsboro, Oreg.). Low voltage imaging was performed without sputter-coating using the Hitachi SU8230 while high voltage imaging was performed on samples sputter-coated with approximately 5 nm gold or gold-palladium. Site-specific milling with the FEI Quanta 3D FEG was performed using a large rough (30 pA) cut to eliminate one cell and fine cut (10 pA) to open the TNT at the gondola. Some images have been pseudo-colored using Adobe Photoshop (Adobe Systems Inc., San Jose, Calif.) and gamma levels adjusted to enhance image contrast and brightness.

### Example 2

### Materials

SILENCER CY3-labeled Negative Control No. 1 siRNA was obtained from Thermo Fisher Scientific, Inc. (Waltham, Mass.). Lipids 1,2-dipalmitoyl-sn-glycero-3-phosphocholine (DPPC); 1,2-di-O-octadecenyl-3-dimethylammonium propane (DODMA); cholesterol; and 1,2-distearoyl-sn-glycero-3-phosphoethanolamine-N-[methoxy(polyethylene glycol)-2000] (ammonium salt) DSPE-PEG(2000) were from Avanti Polar Lipids (Alabaster, Ala.).

### SiRNA Loaded LC-MSN

Dual amine-functionalized mesoporous silica nanoparticles and Dylight 633-functionalized mesoporous silica nanoparticles were incubated with Cy3-siRNA in water for 10 minutes. In parallel, a liposome suspension was prepared by sonicating a mixture of vacuum-dried lipids (DPPC/DODMA/cholesterol/DSPE PEG2000 in 70/10/12/8 mol ratio) in PBS for 15 minutes in a bath sonicator. The liposome suspension was then added to the siRNA-MSN vial and the whole mixture was sonicated for five seconds. The lipid-coated MSN (LC-MSN) were isolated by centrifugation (5 minutes, 21 Krcf) and washed once by PBS. The supernatants were saved for siRNA loading quantification.

Assembly of siRNA-LC-MSN-Loaded Silicified Cells

4T1-GFP breast cancer cells were incubated with siRNA-LC-MSN for six hours at 37° C. Tumor cells were then washed, cryo-silicified, and then surface-coated with TLR ligands PEI, CpG, and MPL.

### In Vitro DC Internalization of NP-Loaded Silicified Cells

To verify BMDC internalization of nanoparticle-drug loaded silicified cells, BMDC were seeded in six-well plates at a density of 5×10⁵ cells per well and the next day, nanoparticle-loaded silicified vaccine cells were added to the cell cultures. The next day, BMDC were collected using 3 mM EDTA. BMDC were then washed with PBS and fixed with 4% paraformaldehyde for 15 minutes at room temperature followed by storage at 4° C. until analysis. BMDC uptake of silicified cells was quantified using flow cytometry (ATTUNE NXT, Thermo Fisher Scientific, Inc., Waltham, Mass.).

The complete disclosure of all patents, patent applications, and publications, and electronically available material cited herein are incorporated by reference in their entirety. In the event that any inconsistency exists between the disclosure of the present application and the disclosure of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A silicified cell comprising:
a silicified cell or silicifiable compartment thereof comprising a surface;
a nanoparticle comprising a bioactive agent loaded into the silicified cell or silicifiable compartment thereof; and
an immunomodulatory moiety bound to the surface.

2. The silicified cell of claim 1, wherein the cell is a tumor cell.

3. The silicified cell of claim 2, wherein the silicifiable compartment of the tumor cell is a cell-derived body or vesicle.

4. The silicified cell of claim 1, wherein the cell is a bacterial cell.

5. The silicified cell of claim 4, wherein the silicifiable compartment of the bacterial cell is a cell-derived body or vesicle.

6. The silicified cell of claim 1, wherein the cell is an embryonic cell, a fetal cell, or a pluripotent stem cell.

7. The silicified cell of claim 6, wherein silicifiable compartment of the embryonic cell, fetal cell, or pluripotent stem cell is a cell-derived body, vesicle, or is part of a spheroid or organoid.

8. The silicified cell of claim 1, wherein the cell is a virus.

9. The silicified cell of claim 1, wherein the immunomodulatory moiety is bound to the surface via a cationic layer disposed on at least a portion of the surface.

10. The silicified cell of claim 1, wherein the immunomodulatory moiety is bound to the surface via an anionic layer disposed on at least a portion of the surface.

11. The silicified cell of claim 1, wherein the immunomodulatory moiety is bound to the surface via siloxane disposed on at least a portion of the surface.

12. The silicified cell of claim 1, wherein the nanoparticle comprises a chemokine, a cytokine, a growth factor, a chemotherapeutic, an anti-angiogenic factor, an antibody, a DAMP, a PAMP, a DNA plasmid, an siRNA, or an mRNA.

13. The silicified cell of claim 1, wherein the immunomodulatory moiety comprises a pathogen-associated molecular pattern (PAMP), a danger-associated molecular molecule (DAMP), a cytokine, or an antibody.

14. The silicified cell of claim 13, wherein the PAMP comprises lipopolysaccharide (LPS), monophosphoryl lipid A (MPL), CpG, R-848, or PolyIC.

15-22. (canceled)

23. A method of inducing an immune response against a cell or a silicifiable compartment thereof, the method comprising:
obtaining a cell or a silicifiable compartment thereof;
loading the cell or a silicifiable compartment thereof with a nanoparticle comprising a bioactive agent;
silicifying the cell or silicifiable compartment thereof, thereby producing an immunogenic silicified cell; and
administering the immunogenic silicified cell to a subject in an amount effective to induce the subject to produce an immune response directed against the cell.

24. The method of claim 23, wherein the nanoparticle includes an agent that blocks immune suppression.

25. The method of claim 24, wherein the agent that blocks immune suppression comprises an anti-TGF-β antibody or an anti-IL-10 antibody.

26. The method of claim 24, wherein the agent that blocks immune suppression comprises an immune checkpoint inhibitor.

27. The method of claim 26, wherein the immune checkpoint inhibitor comprises an anti-PD-1 antibody, an anti-PD-L1 antibody, an anti-TB/13 antibody, or an anti-CTLA-4 antibody.

28-39. (canceled)
